(19) United States
(12) Patent Application Publication
(10) Pub. No.: US 2002/0102237 A1
(43) Pub. Date: Aug. 1, 2002

(54) Compositions and methods for hepatitis treatment

(76) Inventor: Milton Hammerly, (US)

Correspondence Address: Western Patent Group, 6020 Tonkowa Trail, Georgetown, TX, 78628, US

(21) Appl. No.: 10/045,878

(22) Filed: Nov. 7, 2001

Related U.S. Application Data
(60) Provisional application No. 60/246,992, filed on Nov. 13, 2000.

(51) Int. Cl. A61K 38/21; A61K 35/78; A61K 31/7056

(52) U.S. Cl. 424/85.5; 424/764; 514/43

(57) ABSTRACT

Provided herein are medicinal compositions of matter that comprise an anti-hepatitis medication component and a component which contains compounds found in the extract of the milk thistle plant, Silybum marianum. Through use of the compositions of the invention a heretofore unobserved synergy is observed in the treatment of chronic infectious hepatitis in which the liver of a patient treated with a composition according to the invention is protected from degradation, which in many cases may prolong the life of the patient and aid in the patient's recovery from hepatitis by strengthening liver functions. The invention also provides procedures for administering the composition to patients afflicted with hepatitis.

This application claims the benefit of U.S. Provisional Application No. 60/246,992 filed Nov. 13, 2000 the entire contents of which are herein incorporated by reference.

### TECHNICAL FIELD

The present invention relates to medicinal compositions of matter and methods useful for the treatment of various forms of hepatitis. More particularly, it relates to compositions comprising an anti-hepatitis agent in combination with the extract of the plant species Silybum marianum, also commonly referred to as “milk thistle”, and methods for treatments containing such combinations. A composition according to the invention is suitable for oral administration or may be administered to a patient by any other conventional means of drug administration.

### BACKGROUND

Hepatitis, a disease of the human liver, has become a public health matter of epidemic proportions. It is manifested with inflammation of the liver and is usually caused by viral infections and sometimes from toxic agents. Hepatitis may progress to liver cirrhosis, liver cancer, and eventually death. Several viruses such as hepatitis A, B, C, D, E and G are known to cause various types of viral hepatitis. Among them, HBV and HCV are the most serious. UBV is a DNA virus with a virion size of 42 nm. HCV is a RNA virus with a virion size of 30-60 nm. See D. S. Chen, J. Formos. Med. Assoc., 95 (1), 6-12 (1996).

Approximately 200 million individuals world wide may be chronically infected with hepatitis B viral infection (HBV). These infected individuals represent a catastrophic health risk individually, but further represent a major threat of infectivity and magnification of this global epidemic. In the United States 0.1 to 0.5% of all normal, healthy blood donors are hepatitis B surface antigen (BBsAG) positive. In a selected population of individuals at high risk for infection (health care professionals, hemophiliacs, hemodialysis patients, and needle-using drug addicts), the percentage of HBsAg positivity may be as high as 30 percent. Chronic HBV carriers may transmit infection by blood contact or sexual contact. Some HBV carriers are of special concern, especially dentists, physicians, or other health care professionals, which have been documented to spread infection to multiple patient contacts. While hepatitis B viral infection is typically self limited, it can result in a chronic infective state. Ten percent of all HBV infections may result in a chronic viral infection, including: (1) chronic persistent infection and, (2) chronic active infection. The former (chronic persistent infection) often renders the patient asymptomatic or mildly symptomatic but still infectious. The latter (chronic active infection) carries a significant risk of severe debilitation as well as life-threatening manifestations such as cirrhosis, portal hypertension, and death. Hepatocellular carcinoma is also strongly correlated with chronic HBV infection.sup.2-4. Symptoms experienced by patients with chronic HBV infection vary from none to mild degrees of fatigue, malaise, myalgia, arthralgia, and pruritis to more severe manifestations of liver dysfunction (such as bleeding disorders, ascites, and encephalopathy). Biochemical abnormalities associated with chronic HBV include an elevation of liver transaminases in the serum, a reduction in serum albumin, elevation of serum bilirubin and prolongation of the prothrombin time (in advanced end-stage liver disease).

Hepatitis B virus infection leads to a wide spectrum of liver injury. Moreover, chronic hepatitis B infection has been linked to the subsequent development of hepatocellular carcinoma, a major cause of death. Current prevention of HBV infection is a hepatitis B vaccination which is safe and effective. However, vaccination is not effective in treating those already infected (i.e., carriers and patients). Many drugs have been used in treating chronic hepatitis B and none have been proven to be effective, except interferon.

Hepatitis C is difficult to treat, and it is estimated that there are 500 million people infected with it worldwide (about 15 times those infected with HIV). No effective immunization is currently available, and hepatitis C can only be controlled by other preventive measures such as improvement in hygiene and sanitary conditions and interrupting the route of transmission. At present, the only acceptable treatment for chronic hepatitis C is interferon which requires at least six (6) months of treatment and or ribavirin which can inhibit viral replication in infected cells and also improve liver function in some people. Treatment with interferon with or without Ribavirin however has limited long term efficacy with a response rate about 25%. Recent knowledge of the gene structure of hepatitis C virus (HCV) has indicated that it is a single stranded RNA virus having a full length of about 9.5 kilobases (Choo et al. Science, Vol. 244, pp. 359-362 (1989)). Because HCV is a virus very likely to mutate and is not well understood how it replicates, the development of vaccines or anti-HCV drugs is still very slow. Studies have shown that hepatitis C infection is associated with the development of advanced liver disease (Tong et al. N Engl J Med 332: 1463-1466, (1995)) and hepatocellular carcinoma (Liang et al. Hepatology 18: 1326-1333, (1993) and Tsukuma et al. N Engl J Med 328:1797-1801, (1993)), and liver failure due to hepatitis C infection is the most common indication for liver transplantation in many centers (Ascher et al. Hepatology 20:24-27, (1994)). While many patients complain of fatigue (Davis et al. N Engl J Med 321: 1501-1506, (1989)), a number of extrahepatic conditions have been associated with hepatitis C infection such as cryoglobulinemia (Lunel et al. Gastroenterology 106:1291-1300, (1994) and Misiani et al. N Engl J Med 330:751-756, (1994)), porphyria (DeCastro et al. Hepatology 17:551-557, (1993)0, keratitis (Wilson et al. (letter). N Engl J Med 329:62, (1993)) and autoimmune diseases (Pawlotsky et al. Hepatology 19:841-848, (1994)). Currently, the only approved treatment for hepatitis C infection is alpha-interferon. Utilizing the standard therapy of 3 million units subcutaneously three times weekly for six months, approximately 50% respond with normalization of alanine aminotransferase (ALT) (Ascher et al. Hepatology 20 (suppl):24-27, (1994)). Unfortunately, half of those responding relapse upon discontinuation of therapy (Davis et al. N Engl J Med 321:1501-1506, (1989) and Di Bisceglie et al. N Engl J Med 321:1506-1510, (1989)). Most who are retreated will again relapse after drug withdrawal (Tine et al. J Hepatol 13:192-199, (1991)).

Treatment of HCV and HBV with interferon has limited success and has frequently been associated with adverse side effects such as fatigue, fever, chills, headache, myalgias, arthralgias, mild alopecia, psychiatric effects and associated disorders, autoimmune phenomena and associated disorders and thyroid dysfunction.

One current treatment for autoimmune hepatitis requires prolonged administration of corticosteroids such as prednisone and prednisolone. While corticosteroid therapy has been shown to extend life, improve biochemical abnormalities and enhance quality of life in many patients, the beneficial effects of corticosteroids are offset by the often serious complications and side effects associated with the prolonged treatment therewith.

The importance of liver function to the overall health of the body is often overlooked. The liver regulates carbohydrate metabolism by turning glucose into glycogen for storage in the liver. Liver glycogen can release glucose into the blood to maintain blood sugar levels if needed. If the body is low in carbohydrates, the liver can manufacture more carbohydrates from fat or proteins. The liver has storage functions, for storing glycogen, vitamin A, vitamin D, many of the B complex vitamins, iron, and copper. The liver regulates protein metabolism, and manufactures many body proteins such as albumin and blood-clotting factors such as prothrombin and fibrinogen that cause the blood to clot when needed. It makes sex hormone binding globulin which is the protein that binds the steroid sex hormones. The liver makes many proteins for purpose of transporting substances such as fats, iron, hormones, and drugs, around the blood stream, such as high density lipoprotein which transports cholesterol out of the blood-vessel walls back to the liver for excretion. The liver also detoxifies many toxic substances, by the process of conjugation, which involves the addition of a chemical substance to a toxin for its elimination or deactivation. The liver metabolizes or biotransforms drugs, steroid hormones, and waste products of the body such as toxic ammonia. The Kupffer cells engulf and ingest dead cells, cancer cells yeasts, viruses, bacteria, parasites, artificial chemicals, incompletely digested or denatured proteins and dangerous foreign particles.

Thus, it is clear that a stress on a portion of the function of the liver may have profound adverse effects on various health aspects of a patient undergoing any therapy in which a chemical substance is introduced into the bloodstream. However, few workers in the prior art have recognized or addressed the need to protect the liver against chemical toxins during administration of therapeutic agents. U.S. Pat. 6,136,316 teaches hepatoprotective compositions and composition for treatment of conditions related to hepatitis B and E infection using compositions which comprises extracts of plants Rheum emodi Wall., Phyllanthus amarus Linn., Eclipta alba Hassk., Andrographis paniculate Nees., and Picrorhiza kurroa Royle ex Benth. wherein the amount of each of the extracts of the plants Rheum emodi Wall., Phyllanthus amarus Linn., Eclipta alba Hassk., Andrographis paniculate Nees., and Picrorhiza kurroa Royle ex Benth, in the composition is in the range of from 25 to 250 mg.

U.S. Pat. 6,126,942 teaches a medicinal composition for treating hepatitis comprising effective amounts of the herbs Salvia miltiorrhiza root, Polyporus umbellatus root, Poria cocos root, Artemisia capillaries Thunb. plant, Taraxacum mongolicum plant, Paeonia lactiflora root, Panax pseudoginseng root, Bupleurum falcatum root, Crataegus pinnatifida fruit, Curcuma longa tuber, Glechoma longituba plant, Astragalus membranaceus root, Codonopsis pilosula root, Loranthus parasiticus stem, Lycium barbarum fruit, Polygonum cuspidatum root, Zizyphus jujuba fruit, Gentiana manshurica plant and Glycyrrhiza uralensis root.

Regardless of whichever available treatment regimen is ultimately selected by a physician for their patient, current therapeutic approaches do nothing to reduce the chronic liver inflammation caused by the actions or excretions of different strains of hepatitis viruses, and/or the chemical substances used to treat the underlying hepatitis. Thus, if medications were available which could maintain at least the current level of effectiveness of a particular drug towards combating the known strains of hepatitis while simultaneously inhibiting liver tissue degradation and/or inflammation in an amount sufficient to prolong or eliminate the necessity for liver transplant, such compositions and methods for their use in a treatment program would represent a substantial advance in the art of treating these various maladies.

### SUMMARY OF THE INVENTION

The present invention is concerned with medicinal compositions useful in the treatment of chronic infectious hepatitis. Compositions according to the invention comprise an anti-hepatitis medication component and a milk thistle extract (silymarin) component. The invention is also concerned with a process for treating a human patient who is afflicted with chronic infectious hepatitis which comprises the steps of providing a composition according to the invention and introducing such a composition into the patient's body.

Thus, one aspect of the invention comprises a medicinal composition useful in the treatment of any form of hepatitis formed from components comprising: a) a first component comprising the extract of the berries of the milk thistle plant, and especially its seeds; and b) a second component comprising a drug or drug combination recognized by those skilled in the medical arts as being useful in the treatment of any form of hepatitis.

Another aspect of the present invention includes a process for treating a person afflicted with hepatitis comprising simultaneous administration to such person a combination, which combination includes: a) an effective hepato-protective amount of the extract of the berries of the milk thistle plant; and b) a therapeutically effective amount of a substance recognized as having therapeutic benefit in the treatment of any form of hepatitis (“hepatitis treatment drug”).

### DETAILED DESCRIPTION

The present invention is directed at a compositions of matter which comprise an effective therapeutic amount of any anti-hepatitis medication in combination with an effective hepato-protective amount of milk thistle extract for oral administration by the patient themselves, in one preferred embodiment. In other embodiments, a combination according to the invention may be administered enterally, parenterally, subcutaneously, or intramuscularly. Thus, the number of combinations of agents, and embodiments for use of a combination according to the invention which are embraced by the scope of the invention is large.

### The Milk Thistle Component

A naturally-occurring hepatoprotective substance is one commonly referred to as “silymarin”, which is derived from the plant Silybum marianum , also commonly referred to as “milk thistle”. In addition to silymarin, the seeds of the milk thistle plant also contain the compounds silydianin and silychristin, and all of these are believed to be antihepatotoxic agents. According to the invention, addition of an effective hepato-protective amount of any of these or other hepatoprotective substances found in milk thistle to drugs used in treating hepatitis, which drugs have potential for hepatotoxicity, increases the safety margin of the drug and preserves the lives of patients undergoing treatment with such drugs by inhibiting destruction to liver tissues.

Since hepatoprotective agents are believed to alter liver metabolism, they could in theory alter the therapeutic effect of some medications (foe example, in cases where the biochemical pathway of the therapeutic effect and that of the side effect of a drug are one and the same). In most cases, however, the therapeutic effect and the toxic effect are caused by different mechanisms. In the cases of ribavirin and interferon, for instance, it is believed that the hepatotoxicity is completely separate from the mechanism(s) responsible for their mechanisms of action.

One preferred form of milk thistle for use in combinations prepared in accordance with the present invention is the extract from the milk thistle seed, such as that manufactured by Nutraceutical Corporation for Solaray, Inc., Park city, Utah 84068 and available under the designation “Milk Thistle Extract, 175 mg.” Such material is guaranteed to contain 80% (140 mg.) of silymarin. However, the combinations of the present invention are not limited to such a source of milk thistle, and may come from any grade of milk thistle extract which meets the standards of purity for use in the medical arts.

### Anti-hepatitis Medications

According to a preferred form of the invention, the hepatitis treatment drug may be selected from the group consisting of: interferon alfa-2A, interferon alfa-2B, interferon alfacon-1, peginterferon alfa-2B, ribavirin, or lamivudine.

An anti-hepatitis medication such as interferon indirectly enhances the body's anti-viral immune response, while anti-hepatitis medications such as ribavirin exert a direct anti-viral effect. The use of these two medications in combination with one another can dramatically improve the treatment response in patients with chronic infectious hepatitis. Unfortunately, this combined treatment regimen is often not curative and does nothing to specifically reduce the chronic liver inflammation which causes scarring, disease progression and ultimately (in the absence of liver transplantation) death. Milk thistle extract reduces and in some cases prevents liver inflammation. Therefore the anti-viral medications (direct or indirect) and milk thistle extract (silymarin) complement each other by different, mutually-compatible mechanisms of action. The use of such novel combination to treat chronic infectious hepatitis is anticipated to decrease disease progression, reduce the need for liver transplantation and lower the death rates of affected patients.

### Combinations According to the Invention

The present invention is concerned with physical mixtures of an extract of milk thistle and an anti-hepatitis medication. A mixture according to the invention may be employed by itself as an intermediate to which other therapeutically-active materials may be added, or they may be used alone as therapeutic agents. Other therapeutically active materials may include a second anti-hepatitis medication, vitamins, colorants, binding agents, etc. Alternatively, an elixir may be made containing milk thistle extract, an anti-hepatitis medication, and a solvent in which both are soluble, such as ethanol, and adjusted to any desired concentration of the constituents.

To make a composition according to the invention, one selects the desired amounts of each component and physically mixes the two components, using mixing means known to those skilled in the pharmacy art, which may include, without limitation, mechanical mixing. In one preferred embodiment, the mixing is conducted at a temperature in the range of about 25 degrees centigrade to 40 degrees centigrade, with about 40 degrees centigrade being most preferred during the mixing process, and the mixture is cocomminutated until a uniform mixture results. By combining an anti-hepatitis medication with milk thistle extract, a new product composition is provided which both acts on the hepatitis in a therapeutically-beneficial way while protecting the liver from damage. The result is a clinically safe product that is clinically more effective in the long term than compositions of the prior art used to treat hepatitis by virtue of its hepato-protective effects, well tolerated by nearly all patients, and very reasonably priced.

The dose of milk thistle extract required depends upon the hepatoxicity of the drug to be used in combination with the hepatoprotectant (milk thistle extract), and is readily determinable by one of ordinary skill in the art. When interferon and ribavirin are delivered, their usual dose for an adult person varies upon the specific disease being treated. In the cases of the interferons (alfa 2A and alfa 2B), typical doses vary from about 1 million International Units to about 36 million International Units on a daily basis, depending upon factors known to physicians. The recommended dosages for such anti-hepatic medications are well known in the art and can be found in the Physicians Desk Reference, 2000 edition, the entire contents of which is herein incorporated by reference thereto.

According to the invention, the preferred amount of milk thistle extract to be present in a combination according to the invention comprising ribavirin, an interferon, or other anti-hepatic preparation to be administered, is a sufficient amount to provide an effective hepatoprotective amount of silymarin on an average daily basis to the patient. According to another form of the invention, the preferred amount of milk thistle extract to be present in a combination according to the invention comprising ribavirin, an interferon, or other anti-hepatic preparation to be administered, is a sufficient amount to provide at least 50 mg of silymarin on an average daily basis to the patient. According to a preferred form of the invention, the preferred amount of milk thistle extract to be present in a combination according to the invention comprising ribavirin, an interferon, or other anti-hepatic preparation to be administered, is a sufficient amount to provide at least 100 mg of silymarin on an average daily basis to the patient. According to another preferred form of the invention, the preferred amount of milk thistle extract to be present in a combination according to the invention comprising ribavirin, an interferon, or other anti-hepatic preparation to be administered, is a sufficient amount to provide at least 300 mg of silymarin on an average daily basis to the patient.

Compositions according to the invention may be ingested or administered either intermittently or chronically on an as-needed basis (depending on the clinical response of the patient with chronic infectious hepatitis).

According to one preferred form of the invention, there is provided a single dose medicament useful for treating hepatitis comprising an anti-hepatitis medication and a milk thistle extract component, wherein said anti-hepatitis medication is selected from the group consisting: of interferon alfa-2A or interferon alfa-2B, and wherein said anti-hepatitis medication is present in any quantity of between 1 million International Units and 36 million International Units, and wherein the quantity of silymarin in said milk thistle extract component is at least 10 milligrams. According to another form of the invention the quantity of silymarin in the milk thistle extract component is at least 50 milligrams.

According to a preferred form of the invention, there is provided a single dose medicament useful for treating hepatitis comprising an anti-hepatitis medication and a milk thistle extract component, wherein said anti-hepatitis medication is ribavirin, present in any quantity of between 200 and 1200 milligrams, and wherein the quantity of silymarin in said milk thistle extract component is at least 10 milligrams. According to another form of the invention the quantity of silymarin in the milk thistle extract component is at least 50 milligrams.

According to the invention, it is desirable to provide medicaments which contain an effective therapeutic amount of an anti-hepatitis drug in combination with a liver protectant or hepato-protectant as such materials may be referred to. The quantities of the anti-hepatitis drugs which are believed to be therapeutically effective quantities are generally well-known accepted quantities in the field of the medical arts. With regard to the hepato-protective quantity of milk thistle extract, for purposes of this specification and the appended claims the words “hepato-protective amount” when used in reference to the quantity of milk thistle extract, is that amount which is capable of protecting the liver from damage which would otherwise occur in the absence of such milk thistle extract. The amount of milk thistle extract which is hepato-protective is conveniently considered in terms of the hepato-protective constituent of the milk thistle plant, silymarin. An effective hepato-protective amount of milk thistle extract is that quantity sufficient to provide a patient with at least 10 milligrams of silymarin per day. However, since milk thistle extract is believed to be essentially devoid of negative side effects even in large quantities, quantities of 100, 300, or even 600 milligrams of silymarin per patient per day for a patient having a body weight of 70 kg are within the scope of the present invention. The other constituents of the milk thistle extract such as silychristin and silydianin, while present generally in lesser amounts than silymarin in extracts of milk thistle, are also beneficial towards liver protection. For purposes of this invention, “milk thistle extract” means the extract of any portion of the milk thistle plant, including stems, leaves, berries, roots, etc. provided it contains silymarin. The present invention embraces within its scope the use of silymarin, silychristin, and silydianin from plant sources other than milk thistle, or from synthetic routes.

Consideration must be given to the fact that although this invention has been described and disclosed in relation to certain preferred embodiments, obvious equivalent modifications and alterations thereof will become apparent to one of ordinary skill in this art upon reading and understanding this specification and the claims appended hereto. Accordingly, the presently disclosed invention is intended to cover all such modifications and alterations, and is limited only by the scope of the claims which follow.

I claim:

1. A medicinal composition useful in the treatment of chronic infectious hepatitis comprising:
a) an anti-hepatitis medication; and
b) a milk thistle extract component.

2. A composition as set forth in claim 1 wherein said anti-hepatitis medication is selected from the group consisting of: interferon, ribavirin or any derivatives of the foregoing.

3. A composition as set forth in claim 1 wherein said milk thistle extract is any milk thistle extract demonstrated to reduce mammalian liver inflammation either in vitro or in vivo.

4. A composition according to claim 1 wherein said anti-hepatitis medication component is present in any amount between 0.05% and 99.5% by weight based upon total weight of the composition, including every hundredth percentage therebetween.

5. A composition according to claim 1 wherein said milk thistle extract component is present in any amount between 0.05% and 99.5% by weight based upon total weight of the composition, including every hundredth percentage therebetween.

6. A single-dose medicament useful for treating hepatitis comprising a composition according claim 1, wherein said anti-hepatitis medication is selected from the group consisting: of interferon alfa-2A, interferon alfa-2B, or an interferon derivative, and wherein said anti-hepatitis medication is present in a quantity of between 1 million International Units and 36 million International Units, and wherein the quantity of silymarin in said milk thistle extract component is at least 10 milligrams.

7. A single-dose medicament useful for treating hepatitis comprising a composition according claim 1, wherein said anti-hepatitis medication is selected from the group consisting: of interferon alfa-2A, interferon alfa-2B, or an interferon derivative, and wherein said anti-hepatitis medication is present in a quantity of between 1 million International Units and 36 million International Units, and wherein the quantity of silymarin in said milk thistle extract component is at least 300 milligrams.

8. A single-dose medicament useful for treating hepatitis comprising a composition according claim 1, wherein said anti-hepatitis medication is ribavirin or a ribavirin derivative, present in any quantity of between 200 and 1200 milligrams, and wherein the quantity of silymarin in said milk thistle extract component is at least 50 milligrams.

9. A single-dose medicament useful for treating hepatitis comprising a composition according claim 1, wherein said anti-hepatitis medication is ribavirin or a ribavirin derivative, present in any quantity of between 200 and 600 milligrams and wherein the quantity of silymarin in said milk thistle extract component is at least 300 milligrams.

10. A medicinal composition useful in the treatment of chronic infectious hepatitis comprising an anti-hepatitis medication selected from the group consisting of: interferon alfa-2A; interferon alfa-2B; or ribavirin, including derivatives of the foregoing; and further comprising silymarin.

11. A medicinal composition useful in the treatment of chronic infectious hepatitis comprising an anti-hepatitis medication selected from the group consisting of: interferon alfa-2A; interferon alfa-2B; or ribavirin, including derivatives of the foregoing; and further comprising silydianin.

12. A medicinal composition useful in the treatment of chronic infectious hepatitis comprising an anti-hepatitis medication selected from the group consisting of interferon alfa-2A; interferon alfa-2B; or ribavirin, including derivatives of the foregoing; and further comprising silychristin.

13. A method of treating a patient showing symptoms of hepatitis, the method comprising:
administration of a therapeutically effective amount of a hepatitis medication in synergistic combination with a therapeutically effective amount of a hepato-protective agent consisting of milk thistle extract,
wherein the hepatitis medication is administered to alleviate symptoms of hepatitis, and the milk thistle extract is administered to reduce damage to the liver by the action of the hepatitis medication or of the underlying hepatitis.

14. A method according to claim 13 wherein the administration is oral.

15. A method according to claim 13 wherein the administration is parenteral.

16. A method according to claim 13, wherein the hepatitis medication is selected from a group consisting of interferon alfa-2A, interferon alfa-2B, or ribavirin.

17. A method according to claim 13, wherein the hepatitis medication is compounded into a single oral unit with the milk thistle extract.

18. A method according to claim 17, wherein the single oral unit is a tablet.

19. A method according to claim 17, wherein the single oral unit is a capsule.

20. A method according to claim 13, wherein the hepatitis medication is dissolved or suspended in milk thistle extract oil.

21. A method according to claim 20 wherein the dissolved or suspended hepatitis medication is placed in a single oral softgelatin capsule.

22. A method according to claim 13, wherein the hepatitis medication is formed into a first single oral unit, and the milk thistle is formed into a second single oral unit.

23. A method according to claim 22, wherein a number of first single oral units are placed in a first container, and a number of the second single oral units are placed in a second container.

24. The method of claim 23, wherein the first and second containers are packaged together.

25. The method of claim 22, wherein the first and second single oral units are a tablet.

26. The method of claim 22, wherein the first and second single oral units are a capsule.

27. The method of claim 22, wherein the second single oral unit is a softgelatin capsule.

28. The method of claim 13, wherein the hepatitis medication is an interferon being administered orally to the patient in one daily dose of at least 500,000 International Units per day.

29. The method of claim 13, wherein the milk thistle extract is administered orally to the patient in one daily dose containing at least 25 milligrams of any compound selected from the group consisting of: silymarin, silychristin, or silydianin.

30. The method of claim 22, wherein binders and carriers are used in the first and second oral units to facilitate the administration of the hepatitis medication and the milk thistle extract.

31. A process for treating chronic infectious hepatitis comprising the steps of:
a) providing a comprising:
i) an anti-hepatitis medication; and
ii) a milk thistle extract component, and
b) introducing said composition into a human body that is afflicted with a chronic infectious hepatitis.
